(19) United States
(12) Patent Application Publication
(10) Pub. No.: US 2005/0245876 A1
(43) Pub. Date: Nov. 3, 2005

(54) Apparatus and methods for facilitating access through a puncture including sealing compound therein

(75) Inventors: Farhad Khosravi, Los altos Hills, CA (US); Suresh S. Pai, Mountain View, CA (US)

Correspondence Address: Bingham McCuthen, LLP, Suite 1800, Three Embarcadero, San Francisco, CA, 94111-4067, US

(73) Assignee: AccessClosure, Inc., Palo ALto, CA (US)

(21) Appl. No.: 11/112,970

(22) Filed: Apr. 22, 2005

Related U.S. Application Data
(63) Continuation-in-part of application No. 10/745,946, filed on Dec. 24, 2003.

(51) Int. Cl. A61M 5/178

(52) U.S. Cl. 604/164.1

(57) ABSTRACT

Apparatus for delivering a sleeve into a puncture through tissue includes a tubular sheath including a lumen extending between its proximal end and an opening in its distal end. The sleeve includes a hub on a first end thereof disposed adjacent the distal end of the sheath, and a second end extending into the opening and lumen of the sheath. The hub is slidable along an exterior of the sheath for drawing the sleeve out of the opening and along an exterior of the sheath. During use, a guide wire is placed through the puncture into a blood vessel, and sealing compound is introduced into the puncture around the guide wire. The sheath is advanced into the puncture over the guidewire while maintaining the hub adjacent the patient's skin, causing the sleeve to be deployed from the sheath and line the puncture as the sheath is advanced into the puncture.

### RELATED APPLICATION DATA

This application is a continuation-in-part of application Ser. No. 10/745,946, filed Dec. 24, 2003, the entire disclosure of which is expressly incorporated herein by reference.

### FIELD OF INVENTION

The present invention relates generally to apparatus and methods for sealing punctures in a body, to apparatus and methods for facilitating access through a puncture extending through tissue, and, more particularly, to apparatus and methods for delivering a flexible sleeve or other lining into a puncture extending through tissue that includes a sealing compound therein, e.g., to facilitate accessing a vessel or other body lumen via the puncture during a procedure.

### BACKGROUND

Apparatus and methods are known for accessing a patient's vasculature percutaneously for performing a procedure within the vasculature. For example, a hollow needle may be inserted through a patient's skin and overlying tissue into a blood vessel. A guidewire is then passed through the needle into the blood vessel, whereupon the needle is removed. An introducer sheath is then advanced over the guidewire into the vessel, e.g., in conjunction with or subsequent to one or more dilators. A catheter or other device may be advanced through the introducer sheath and over the guidewire into a position for performing a medical procedure within the patient's body. In this manner, the introducer sheath facilitates introducing various instruments into the vessel, while minimizing trauma to the vessel wall and blood loss.

Upon completing the procedure, the instrument(s) and introducer sheath are removed, leaving a puncture extending between the skin and the vessel. To seal the puncture, external pressure may be applied to the overlying tissue, e.g., manually and/or using sandbags, until hemostasis occurs. This procedure, however, can be time consuming and expensive, requiring as much as an hour of a medical professional's time. It is also uncomfortable for the patient, and may require the patient to remain immobilized in an operating room, catheter lab, or holding area. In addition, a risk of hematoma exists from bleeding before hemostasis occurs.

Various apparatus and methods have been suggested for sealing a percutaneous puncture instead of or in addition to using external pressure. For example, U.S. Pat. No. 5,108,421 issued to Fowler, and U.S. Pat. Nos. 5,192,302 and 5,222,974 issued to Kensey et al., describe delivering a collagen plug into a puncture site.

Such sealing methods generally involve introducing plugs or other materials into the puncture after completing the procedure and removing the introducer sheath. With the introducer sheath removed, there is substantial risk of hematoma within the tissue surrounding the puncture as blood from the vessel leaks into the puncture, which may be uncomfortable and/or harmful to the patient. Further, temporary hemostasis devices for isolating the vessel from the puncture may be difficult to use effectively and/or may be expensive. Despite attempts to isolate the vessel from the puncture while delivering a plug or other sealing material, the sealing material may still leak and/or become exposed in the vessel, where the sealing material may risk causing an embolism in the vessel.

### SUMMARY OF THE INVENTION

The present invention is directed to apparatus, systems, and methods for facilitating access through a puncture in a body, e.g., extending from a patient's skin to a blood vessel or other body lumen, and/or for sealing such punctures. More particularly, the present invention includes apparatus and methods for delivering a sleeve into a puncture extending through tissue to a blood vessel or other body lumen and/or for facilitating access through a sealing compound disposed within the puncture.

In accordance with one embodiment, a system is provided that includes a tubular sheath including a proximal end, a distal end sized for insertion into the puncture, and a lumen extending between the proximal end and an opening in the distal end; and a sleeve including first and second ends, a hub on the first end disposed adjacent the distal end of the sheath. The second end of the sleeve may extend into the opening and lumen of the sheath, the hub being slidable along an exterior of the sheath for drawing the sleeve out of the opening and along the exterior of the sheath.

In addition, the system may include a guidewire, and an assembly for delivering a sealing compound into the puncture around the guidewire. In one embodiment, the assembly may include a delivery sheath and a source of sealing compound for delivering sealing compound through the delivery sheath.

In accordance with another embodiment, a method is provided for lining a puncture extending from a patient's skin to a body lumen using a tubular sheath and a thin-walled sleeve disposed within a lumen of the tubular sheath. The sleeve may include a hub on a first end thereof adjacent a distal end of the sheath and a second end disposed within the lumen of the sheath.

A guide wire may be placed through the puncture from the patient's skin into the body lumen, and a sealing compound may be introduced into the puncture, e.g., around at least a portion of the guide wire. The hub of the sleeve may be placed adjacent the patient's skin, and the sheath may be advanced into the puncture over the guidewire while maintaining the hub adjacent the patient's skin. This causes the sleeve to be deployed from the lumen of the tubular sheath and cover an exterior of the sheath to line the puncture as the sheath is advanced into the puncture.

In accordance with yet another embodiment, a system is provided that includes an elongate occlusion member including a proximal end, a distal end having a size and shape for insertion into the puncture, and an expandable occlusion element on the distal end. A thin-walled sleeve may extend along an exterior of the occlusion member from the proximal end towards the occlusion element, the sleeve being separable from the exterior of the occlusion member. The system may also include a delivery sheath advanceable over the occlusion member and sleeve for delivering a sealing compound into the puncture around at least a portion of the sleeve.

In addition or alternatively, the system may also include an introducer or procedure sheath including a proximal end, a distal end sized for insertion into the puncture, and a lumen extending between the proximal and distal ends for delivering one or more instruments into the body lumen. The distal of the introducer sheath may be advanceable through the sleeve after removing the occlusion member from the puncture or between the sleeve and the occlusion member.

In accordance with still another embodiment, a method is provided for delivering a sealing compound into a puncture extending from a patient's skin to a body lumen. An elongate member may be introduced from the patient's skin through the puncture into the body lumen, the elongate member including a flexible thin-walled sleeve extending along an exterior of the elongate member. A sealing compound may be delivered into the puncture, the sealing compound at least partially surrounding the elongate member and sleeve.

The body lumen may then be accessed through the sleeve For example, an introducer or procedure sheath may be advanced through the sleeve into the puncture. The introducer sheath may be advanced between the sleeve and elongate member into the puncture, or the elongate member may be removed from the puncture while the sleeve remains within the puncture, whereupon the introducer sheath may be advanced through the sleeve.

Other aspects and features of the invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a side view of a system for sealing a puncture, including an introducer sheath carrying an everting sleeve, an occlusion member, a delivery sheath, and a syringe assembly for delivering sealing compound via the delivery sheath.

FIG. 1A is a side view of the occlusion member of FIG. 1, with an occlusion element thereon in an expanded condition.

FIG. 2 is a cross-sectional view of the introducer sheath of FIG. 1, taken along line 2-2.

FIGS. 3A and 3B are cross-sectional side views, showing a method for loading an everting sleeve into a tubular member.

FIGS. 4A-4C are cross-sectional views of a patient's body, illustrating a method for delivering a sealing compound a puncture extending between the patient's skin and a blood vessel.

FIG. 5A-5C are cross-sectional views of a patient's body, showing a method for delivering a sleeve into the puncture of FIGS. 4A-4E after delivering a sealing compound therein.

FIG. 6 is a side view of another system for delivering a sleeve into a puncture extending through tissue.

FIGS. 7A-7D are cross-sectional views of a patient's body, showing a method for delivering a sleeve into the puncture using the system of FIG. 6.

### DETAILED DESCRIPTION OF THE ILLUSTRATED EMBODIMENTS

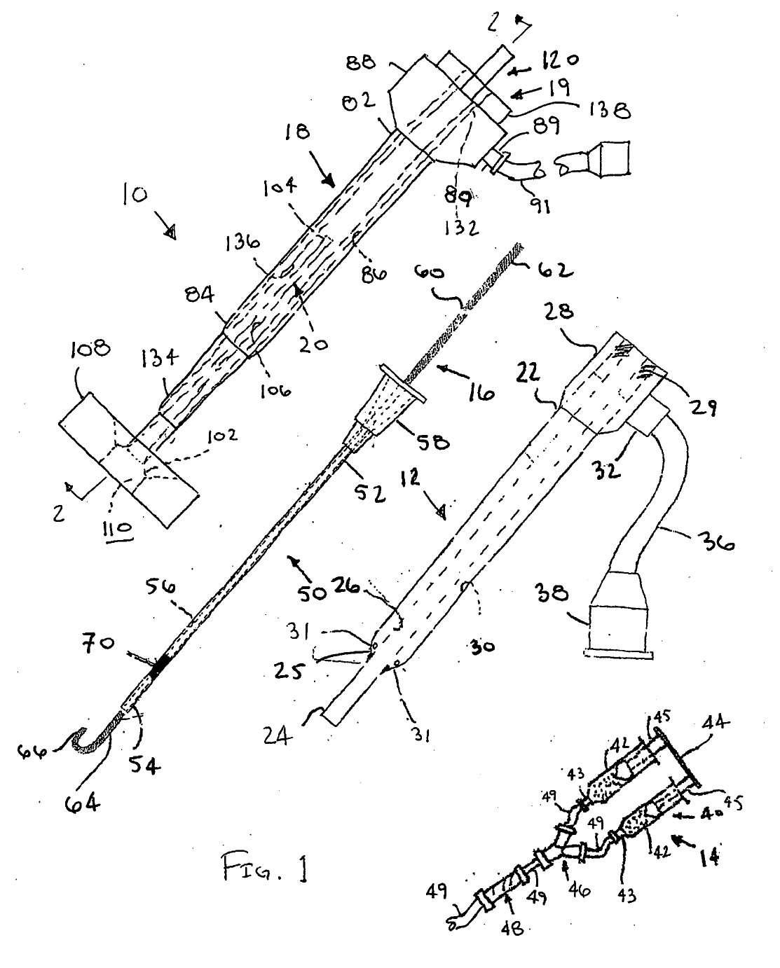

Turning to the drawings, FIG. 1 shows an exemplary embodiment of a system 10 for delivering sealing compound into a puncture through tissue, e.g., a percutaneous puncture for accessing a blood vessel or other body lumen (not shown), and/or for accessing the body lumen via the puncture. Generally, the system 10 includes a delivery or injection sheath 12, a source of sealing compound 14, an occlusion member 16, and an introducer or procedure sheath 18 carrying a dilator 19 and an everting sleeve 20. Optionally, the system 10 may include other components, e.g., one or more of a needle for creating the puncture, a guidewire, and/or one or more sections of tubing (not shown). In addition or alternatively, the system 10 may include other or further components for creating the puncture, introducing the delivery sheath 12 and/or guidewire into a body lumen, and/or accessing the vessel, e.g., for introducing instruments into the vessel via the puncture.

Generally, the delivery sheath 12 is an elongate tubular member, including a proximal end 22, a distal end 24, and a primary or guidewire lumen 26 extending between the proximal and distal ends 22, 24. In addition, the delivery sheath 12 may include one or more secondary or injection lumens 30 that extend from the proximal end 22 to one or more outlets 31 (e.g., two, as shown) in the wall of the delivery sheath 12.

As shown, a single secondary lumen 30 is disposed concentrically around the primary lumen 26. Alternatively, one or more secondary lumens (not shown) may be formed or otherwise provided in the wall of the delivery sheath 12, e.g., in a side-by-side arrangement. In a further alternative, a delivery sheath including a single lumen (not shown) may be provided. The primary lumen 26 may be of sufficient size to accommodate sliding a guidewire therethrough, e.g., between about 0.014 and 0.018 inch (0.35-0.45 mm), while the secondary lumen 30 may be of sufficient size to accommodate delivering sealing compound therethrough.

The secondary lumen 30 extends from a housing 28 on the proximal end 22 of the delivery sheath 12 to an intermediate portion 25 between the proximal and distal ends 22, 24. As shown, the intermediate portion 25 tapers where the secondary lumen 30 terminates, with the delivery sheath 12 having a smaller diameter from the intermediate portion 25 to the distal end 24 (e.g., since only the primary lumen 26 extends along this portion of the delivery sheath 12). The smaller diameter distal portion may have a desired length, e.g., at least about five millimeters (5 mm). The outlet(s) 31 may be provided on the intermediate portion 25, e.g., where the delivery sheath 12 tapers, which may facilitate directing the sealing compound delivered through the secondary lumen 30 radially outwardly away from the delivery sheath 12.

The housing 28 may be attached to or otherwise provided on the proximal end 22 of the delivery sheath 12. The housing 28 may include one or more side ports 32 (one shown) that communicate with an interior of the housing 28 and the secondary lumen 30 of the delivery sheath 12. The housing 28 may include one or more seals 29 to seal the interior of the housing 28 such that sealing compound delivered from the side port 32 may be directed through the secondary lumen 30. Optionally, the housing 28 may also include one or more seals (not shown), e.g., a hemostatic seal, for sealing the primary lumen 26 while accommodating inserting a guidewire or other instrument (not shown) into the lumen 26, e.g., preventing body fluids, such as blood, from escaping proximally through the delivery sheath 12, as is known in the art.

A section of flexible tubing 36 may be connected to or otherwise extend from the side port 32 to a luer lock adapter 38, a manual shut-off valve (not shown), and/or other connector (also not shown), e.g., to facilitate connecting tubing and the like (also not shown) to the side port 32. A source of sealing compound, such as the dual-syringe assembly 40 described below, may be connected to the luer lock adapter 38.

In alternative embodiments, the delivery sheath may be a tubular member including a single lumen (not shown), which may include a hub, side port, and/or other components similar to the embodiment described above. Additional information on such delivery sheaths and methods for using them may be found in co-pending application Ser. Nos. 10/454,362, filed Jun. 4, 2003 and 10/745,946, filed Dec. 24, 2003. The entire disclosures of these references and any others cited therein are expressly incorporated herein by reference.

Turning to FIGS. 1 and 1A, the occlusion member 16 includes a guidewire or other elongate member 60 carrying a tamp or other occlusion element 70. Optionally, the occlusion member 16 may also include a retaining sheath or other constraint 50 slidable over the guidewire 60, e.g., for maintaining the tamp 70 in a contracted condition. For example, the retaining sheath 50 may be an elongate tubular member including proximal and distal ends 52, 54, and a lumen 56 extending therebetween. A hub 58 may be located on the proximal end 52, e.g., to facilitate manipulating the retaining sheath 50. The retaining sheath 50 may have a diameter or other size to allow the distal end 54 to be inserted into and/or through the primary lumen 26 of the delivery sheath 12, while the hub 58 may be larger than the size of the primary lumen 26, e.g., to provide a stop limiting distal advancement of the retaining sheath 50 into the delivery sheath 12. The retaining sheath 50 may be sufficiently flexible to conform to the surrounding anatomy, e.g., when the retaining sheath 50 is inserted into or removed from a puncture, e.g., along with other components, such as the guidewire 60.

As best seen in FIG. 1A, the guidewire 60 may be an elongate member including a proximal end 62 and a distal end 64, e.g., including a “J” tip 66. The guidewire 60 may be formed from a solid wire, one or more coiled wires, and/or a solid-walled tube. Optionally, one or more coatings (not shown) may be provided on an interior or exterior surface of the guidewire 60, e.g., to seal the wall of the guidewire and/or to provide a lubricious exterior surface. The guidewire 60 may be formed from a variety of known materials, e.g., metals, such as stainless steel or Nitinol, plastics, and/or composite materials. Thus, the guidewire 60 may be sufficiently flexible to navigate tortuous anatomy, but may have sufficient column strength to be pushable from the proximal end 62.

The tamp 70 may an expandable structure adjacent the distal tip 66 that may be biased towards an enlarged condition (e.g., as shown in FIG. 1A), but may be resiliently compressible towards a contracted condition (e.g., as shown in phantom in FIG. 1). In the embodiment shown, the tamp 70 includes a braided mesh of wires or other fibers 72 that assume a generally spherical or elliptical disk shape in the enlarged condition. The fibers 72 may be formed from a shape memory material, e.g., Nitinol, stainless steel, plastic, and the like, that has the enlarged condition programmed into the fibers 72, e.g., by heat treatment. Thus, the fibers 72 may be elastically (or super-elastically) deformed, e.g., compressed into the contracted condition using the retaining sheath 50, yet resiliently expandable towards the enlarged condition once released, as explained further below. The tamp 470 may shorten as it expands from the contracted condition towards the enlarged condition, and may lengthen again as it is compressed back towards the contracted condition.

The fibers 72 may include a coating, cover, or other skin (not shown) that covers all or a portion of the tamp 70. For example, at least the proximal portion 70a of the tamp 70 may include a coating or other skin that extends across the spaces between the fibers 72 such that the proximal portion 70a is substantially nonporous. Alternatively, all of the tamp 70 may include a coating or other skin.

In another alternative, the tamp may include a plurality of struts (not shown) that are expandable between enlarged and contracted conditions. The struts may extend substantially axially in the contracted condition and may buckle at an intermediate location thereon as they expand radially outwardly towards the enlarged condition. The struts may be biased towards the enlarged condition (similar to the mesh above), or may be selectively expanded and/or compressed, e.g., using an internal pull wire or other actuator (not shown). In another alternative, the occlusion member 16 may include an elongate tubular member carrying a balloon (not shown), such as those described below or disclosed in co-pending application Ser. No. 10/454,362, filed Jun. 4, 2003, incorporated above, and Ser. No. 10/806,927, filed Mar. 22, 2004, the entire disclosure of which is also expressly incorporated herein by reference.

Returning to FIG. 1, the source of sealing compound 14 may include a dual syringe assembly 40 or other delivery device, e.g., that includes two components of a sealing compound. As shown, the syringe assembly 40 includes a pair of syringe barrels 42, including outlets 43 and a plunger assembly 44 slidable into the barrels 42 to cause the components therein to be delivered through the outlets 43. In the embodiment shown, the plunger assembly 44 includes a pair of plungers 45 coupled to one another that are received in respective barrels 42. In this manner, both plungers 45 may be manually depressed substantially simultaneously to deliver the components together from the syringe barrels 42. Alternatively, a system for automatically advancing the plungers 45 and/or otherwise delivering the components in the barrels 42 may be used, such as those disclosed in co-pending application Ser. No. 10/806,934, filed Mar. 22, 2004, the entire disclosure of which is expressly incorporated herein by reference.

Optionally, the delivery device 14 may include a “Y” fitting 46, a static mixer 48, and/or tubing 49, e.g., for connecting the “Y” fitting 48 to outlets 43 of the barrels 42, the mixer 48 to the “Y” fitting 46 and/or to the side port 32 of the delivery sheath 12, such that the sealing components ejected out of the barrels 42 may mix before being directed into the side port 32 of the delivery sheath 12. The outlets 43, “Y” fitting 46, mixer 48, and/or tubing 49 may include cooperating connectors, e.g., luer lock connectors and the like (not shown), for connecting them together.

Respective sealing components may be provided in each syringe barrel 42 of the syringe assembly 40 that, when mixed together, are activated to form a hydrogel or other sealing compound. Additional information on such hydrogels and systems for delivering them are disclosed in U.S. Pat. Nos. 6,152,943, 6,165,201, 6,179,862, 6,514,534, and 6,379,373, and in co-pending applications publication Nos. 2002-0106409 published on Aug. 8, 2002, 2003-0012734 published on Jan. 16, 2003, 2002-0114775 published on Aug. 22, 2002, and 2004-0249342 published on Dec. 9, 2004. The disclosures of these references and any others cited therein are expressly incorporated by reference herein.

With continued reference to FIG. 1, the introducer sheath 18 is an elongate tubular member including a proximal end 82, a distal end 84, and a lumen 86 extending between the proximal and distal ends 82, 84. The introducer sheath 18 may terminate in a tapered distal tip 85 for facilitating advancing the introducer sheath 18 substantially atraumatically through tissue into a puncture. Exemplary materials for the introducer sheath 18 may include one or more plastics, such as polyvinyl chloride (PVC), FEP, polyimide, polyamide, PEEK, nylon, PET, PEBAX, and polyethylene, metals, such as stainless steel, and nickel titanium, and/or composite materials. The introducer sheath 18 may be substantially rigid, semi-rigid, or substantially flexible, e.g., to facilitate insertion through a puncture into a blood vessel or other body lumen. The introducer sheath 18 may have an outer diameter or other cross-section between about 0.050-0.20 inch (1.25-5.0 mm) and/or a wall thickness between about 0.005-0.015 inch (0.125-0.375 mm).

A housing 88 may be attached to or otherwise provided on the proximal end 82 of the introducer sheath 18. The housing 88 may include a side port 89 that communicates with an interior of the housing 88 and the lumen 86 of the introducer sheath 18. A section of flexible tubing 91 may be connected to or otherwise extend from the side port 89, terminating in a manual shut-off valve and/or a luer lock or other connector (not shown), e.g., to facilitate connecting tubing and the like (not shown) to the side port 89. The housing 88 may also include one or more seals (not shown), e.g., a hemostatic seal, for substantially sealing the lumen 86 of the delivery sheath 18, yet accommodating inserting one or more instruments (not shown) into the lumen 86.

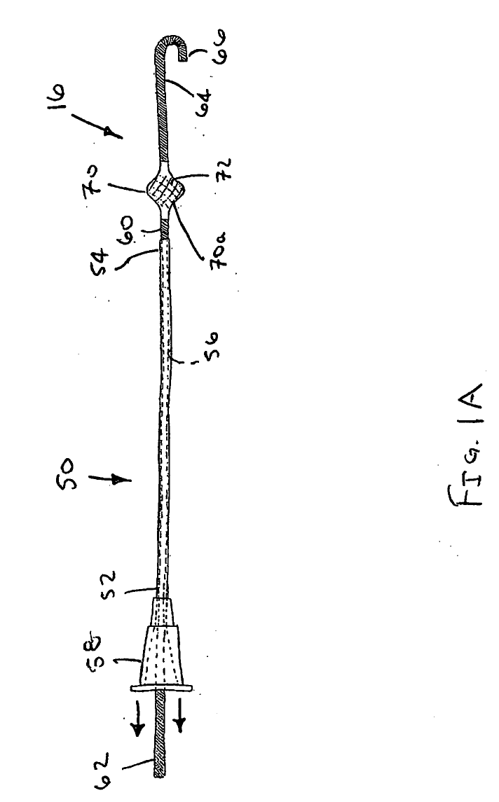

With reference to FIGS. 1 and 2, the sleeve 20 may be a relatively thin-walled substantially flexible tubular member including a first end 102, a second end 104, and a lumen 106 extending between the first and second ends 102, 104. In one embodiment, the sleeve 20 may be a substantially closed-walled tube, e.g., with one or more longitudinal seams (not shown) extending between the first and second ends 102, 104. The longitudinal seam(s) may be substantially permanently fixed or may be weakened or otherwise separable, as described further below.

In an exemplary embodiment, the sleeve 20 may be formed from a substantially inelastic sheet of material whose longitudinal edges are bonded, melted, welded, or otherwise attached to one another, e.g., butted together or lapped one over the other, to form a tubular structure. In one embodiment, the sleeve 20 may be formed from expanded polytetraflouroethylene, e.g., having a wall thickness of between about 0.0001-0.1 inch (0.0025-2.5 mm), e.g., less than about 0.004 inch, and/or less than about 0.001 inch, similar to the membranes described in U.S. Pat. Nos. 5,531,717, 5,676,688, and 6,240,968, the entire disclosures of which are expressly incorporated herein by reference.

Alternatively, the sleeve 20 may be extruded or otherwise formed from a continuous length of flexible tubing. In further alternatives, the sleeve 20 may be formed from other materials, such as Dacron or silk fabrics, fiber meshes, and the like, e.g., with or without coatings to provide a substantially nonporous wall. Such materials may be woven, knitted, or otherwise formed into a tubular shape. In yet another alternative, the sleeve 20 may be formed from a sheet whose longitudinal edges (not shown) are simply lapped over one another between the first and second ends 102, 104 and/or butted against one another. The edges may be bonded together, adhered using an adhesive, connected with threads or other fasteners, and the like.

Optionally, one or both of the inner and outer surfaces of the sleeve 20 may include a lubricious coating, e.g., to reduce friction as the sleeve 20 everts around itself and/or around the distal end 84 of the introducer sheath 82 (or other components), as described further below. As shown, the second end 104 of the sleeve 20 may be open. Alternatively, the second end 104 may be substantially closed, e.g., including a weakened or penetrable end wall (not shown) allowing the second end to be opened by advancing a guidewire or other instrument until it penetrates through the end wall.

An annular hub 108 may be attached to or otherwise provided on the first end 102 of the sleeve 20. The hub 108 may have a generally planar or curved configuration, e.g., to facilitate placing the hub 108 in contact with a patient's skin or other anatomy such that the hub 108 conforms substantially to the anatomy. The hub 108 may include a passage 110 therethrough, e.g., including a frustoconical proximal portion 110a and a cylindrical distal portion 110b. The distal portion 110b may have a diameter or other cross-section larger than the outer diameter of the introducer sheath 18. Thus, the distal end 84 of the introducer sheath 18 may be directed into the passage 110, e.g., guided by the ramped surfaces of the proximal portion 110a into and through the distal portion 110b, as described further below.

As shown in FIGS. 1 and 2, the sleeve 20 may be carried within a tubular member 120 sized to be slidably received within the lumen 86 of the introducer sheath 18. The tubular member 120 may include a proximal end 122, a distal 124, and a lumen 126 extending therebetween, within which the sleeve 20 may be received. The tubular member 120 may be formed from substantially rigid, semi-rigid, or substantially flexible material, similar to the introducer sheath 18.

The distal end 124 of the tubular member 120 may include a blunt, tapered, and/or rounded edge, e.g., to facilitate the sleeve 20 sliding around the distal end 124 of the tubular member 120 during deployment, as described further below. The tubular member 120 may have sufficient length that the proximal end 122 of the tubular member 120 may extend proximally beyond the hub 88 of the introducer sheath 18, while the distal end 124 of the tubular member 120 is disposed adjacent to or beyond the distal end 84 of the introducer sheath 18.

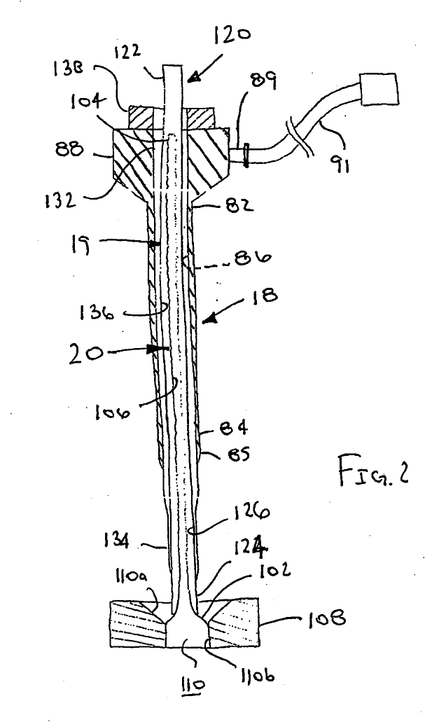

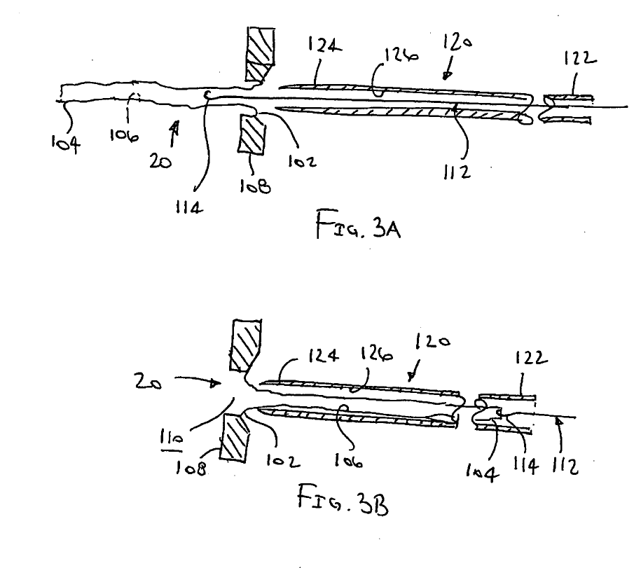

The tubular member 120 may facilitate loading the sleeve 20 into the introducer sheath 18, e.g., during original manufacturing or immediately before a medical procedure. For example, turning to FIGS. 3A and 3B, the sleeve 20 may be provided initially with the second end 104 disposed distally relative to the first end 102. With the hub 108 disposed adjacent the distal end 124 of the tubular member 120, a tool 112, e.g., including an elongated hook 114, forceps, grabber, or other mechanism (not shown), may be inserted into the proximal end 122 of the tubular member, through the lumen 126, and into the first end 102 and lumen 104 of the sleeve 20. The hook 114 (or other mechanism) may engage the second end 104, whereupon the tool 112 may be withdrawn proximally back through the tubular member 120, thereby everting the second end 104 of the sleeve 20 within itself as the second end 104 is pulled into the lumen 126 of the tubular member 120.

Once the second end 104 is disposed within the lumen 126 of the tubular member 120, e.g., adjacent the proximal end 122 of the tubular member, the hook 114 may be disengaged from the second end 104. The hook 114 may then be withdrawn proximally from the tubular member 120. Optionally, the sleeve 20 may be twisted about its longitudinal axis or otherwise compressed before being loaded into the tubular member 120, e.g., to reduce its initial profile and/or facilitate loading.

The tubular member 120 may then be inserted through the introducer sheath 18, e.g., before the introducer sheath 18 is packaged during manufacturing. Alternatively, the tubular member and introducer sheath 18 may be packaged separately or side-by-side in a single package. The proximal end of the tubular member 120 may be inserted into the lumen 86 from the distal end 84 of the introducer sheath 18 until the hub 108 of the sleeve 20 is disposed adjacent to or distal to the distal end 84 of the introducer sheath 18, as shown in FIGS. 1 and 2.

Optionally, as shown in FIGS. 1 and 2, a dilator 19 may also be provided, e.g., within the lumen 86 of the introducer sheath 18. The dilator 19 may include a proximal end 132, a distal end 134 sized for insertion through the lumen 86 of the introducer sheath 18, a lumen 136 extending between the proximal end distal ends 132, 134, and a hub or other handle 138 on the proximal end 132. The distal end 134 may include a tapered or multiple ramped shape, similar to known dilators. The dilator 19 may be formed from substantially rigid, semi-rigid, or substantially flexible materials, similar to the introducer sheath 18.

Similar to the tubular member 120, the dilator 19 may be loaded into the introducer sheath 18 during manufacturing or immediately before a procedure. In addition, the dilator 19 may be loaded into the introducer sheath 18 before or after the tubular member 120, e.g., by inserting the distal end 134 of the dilator 19 into the hub 88 and lumen 86 of the introducer sheath 18 (around the tubular member 120 if already loaded) until the hub 138 abuts or is locked at the hub 88. Once inserted into the introducer sheath 18, the distal end 134 of the dilator 19 may extend beyond the distal end 84 of the introducer sheath 18, e.g., to provide a gradually tapering transition for the assembly. Thus, before a procedure, the sleeve 20, tubular member 120, dilator 19, and introducer sheath 18 may be disposed concentrically around one another in an assembly, as shown in FIGS. 1 and 2. Optionally, one or both of the tubular member 120 and dilator 19 may be eliminated, if desired, and the sleeve 20 may be everted and disposed directly within the lumen 86 of the introducer sheath 18 or the lumen 136 of the dilator 19.

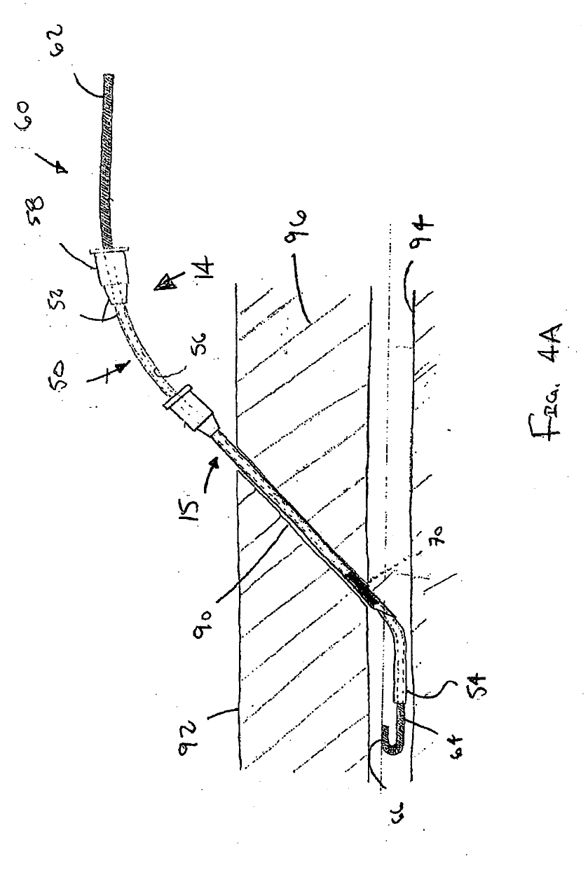

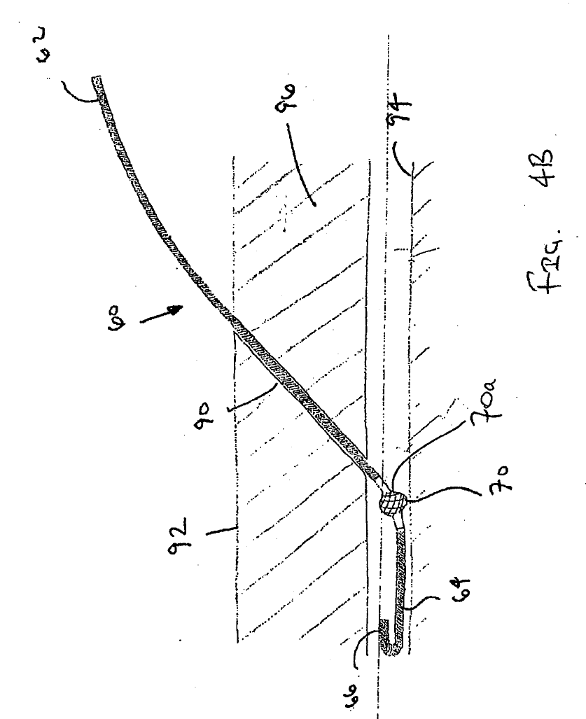

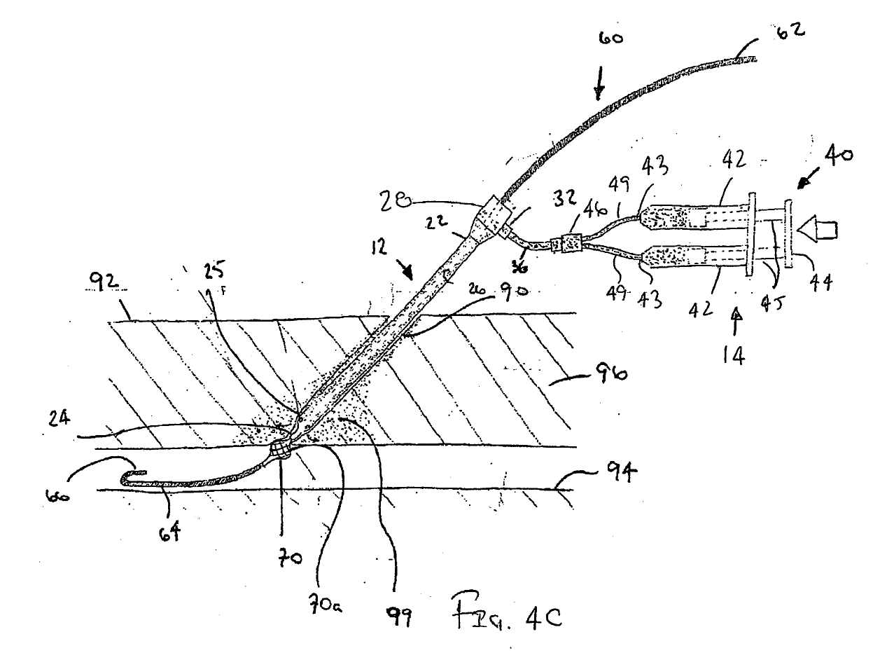

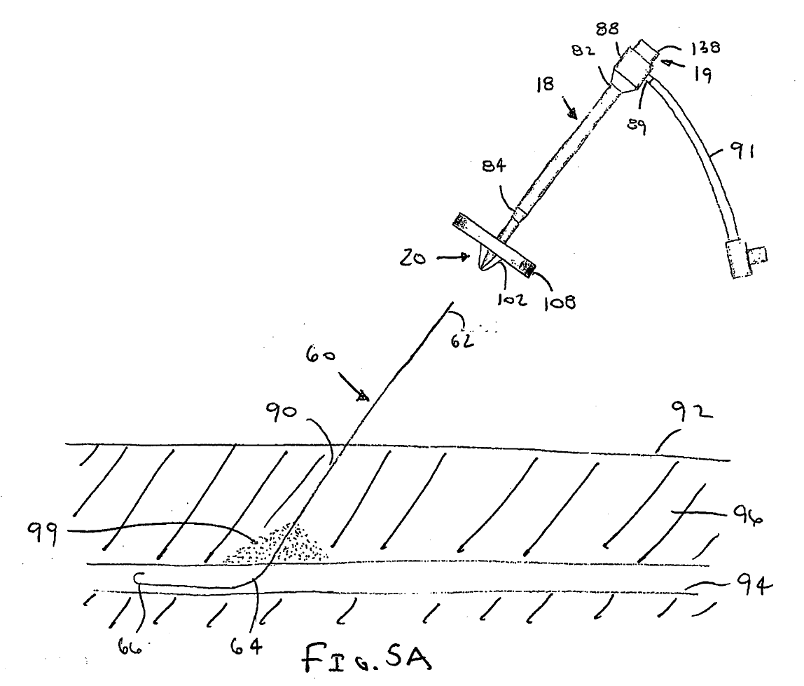

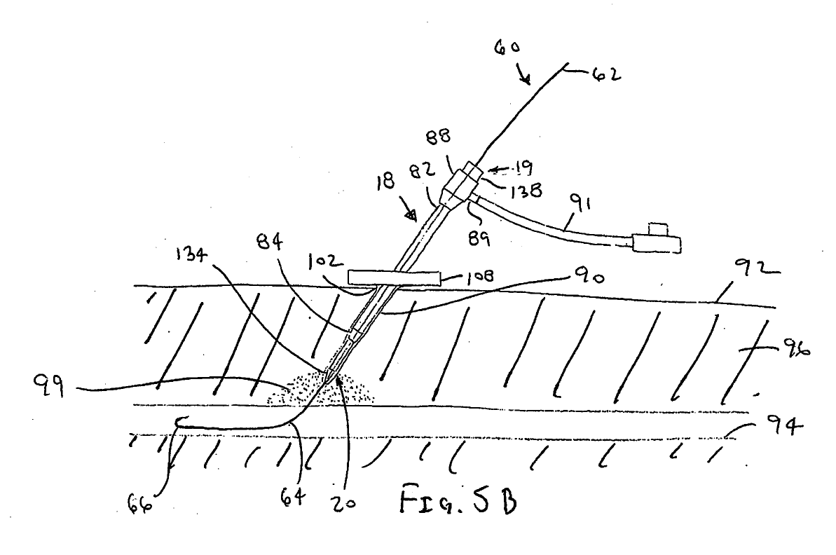

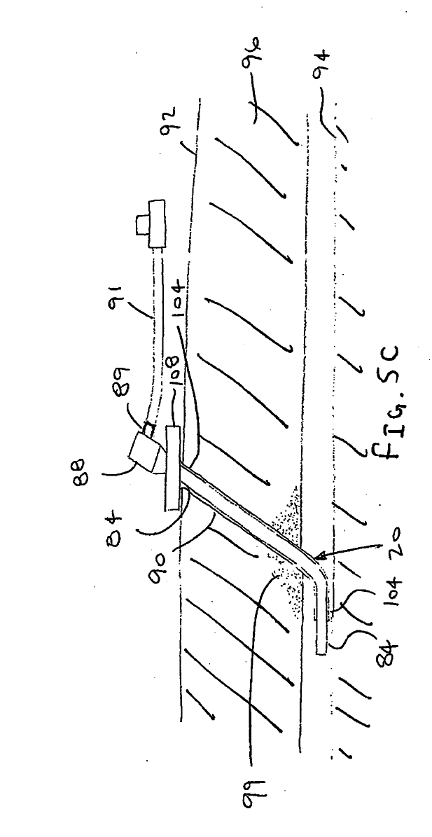

Turning to FIGS. 4A-4E and 5A-5C, a method is shown for delivering an introducer sheath and/or sleeve, such as the introducer sheath 18 and sleeve 20 described above, into a passage extending through tissue 96. In the illustrated embodiment, the passage is a percutaneous puncture 90 extending from a patient's skin 92 to a blood vessel or other body lumen 94. For example, the vessel 94 may be a peripheral artery, e.g., a femoral artery, a carotid artery, and the like. It will be appreciated that systems and methods constructed and undertaken in accordance with various embodiments of the invention may be used to seal other passages through tissue within a patient's body.

Initially, as shown in FIGS. 4A-4C, the puncture 90 may be created and sealing compound 99 may be delivered into the puncture 90. Turning to FIG. 4A, to create the puncture 90, a hollow needle 15 may be inserted through the patient's skin 92 and intervening tissue 96 into the vessel 94. The occlusion member 16, e.g., the guidewire 60 and retaining sheath 50, may be inserted into the puncture 90, e.g., through the needle 15 until the distal tip 66 is disposed within the vessel 94. As shown, the retaining sheath 50 covers the tamp 70 on the guidewire 460 as the guidewire 460 is advanced through the needle 416, thereby maintaining the tamp 70 in the contracted condition.

Turning to FIG. 4B, once the tamp 70 is within the vessel 94, the needle may be removed, and the tamp 70 may be expanded within the vessel 94. For example, the retaining sheath 50 may be retracted completely (or only partially, not shown) out of the puncture 90 to expose the tamp 70, whereupon the tamp 70 may self expand within the vessel 94. Alternatively, the tamp 70 may be selectively expandable, e.g., using an internal pull wire or other actuator (not shown). Thus, once the tamp 60 is exposed within the vessel 94, the tamp 70 may be expanded, e.g., by pulling the pull wire until the tamp 70 attains a desired enlarged size and/or configuration. In another alternative, the tamp 70 may be a balloon or other expandable member (not shown), such as those described in co-pending application Ser. Nos. 10/454,362 and 10/806,927, incorporated by reference above, e.g., that may be inflated using inflation media.

Turning to FIG. 4C, the delivery sheath 12 may be advanced over the guidewire 60 into the puncture 90, e.g., before or after the tamp 70 is expanded. As shown, the delivery sheath 12 may be advanced over the guidewire 60 until the distal end 24 enters the vessel 94. For example, the proximal end 62 of the guidewire 460 may be backloaded through the primary lumen 26 of the delivery sheath 12, and the delivery sheath 12 may be advanced into the puncture 90, the guidewire 60 sliding through the primary lumen 26.

After the tamp 70 is expanded, the guidewire 60 may be partially retracted from the vessel 94, e.g., by pulling the proximal end 62 of the guidewire, until the proximal portion 40a of the tamp 470 contacts the distal end 24 of the delivery sheath 12 (providing a first tactile feedback). The guidewire 60 may then be pulled further until the tamp 70 contacts the wall of the vessel 94 (providing a second tactile feedback), thereby partially in retracting the delivery sheath 12 back into the puncture 90, e.g., until the distal end 24 is disposed adjacent the vessel 94.

Alternatively, the guidewire 60 may be retracted until the tamp 70 contacts the wall of the vessel 94 before the delivery sheath 12 is introduced. The delivery sheath 12 may then be advanced into the puncture 90 until the distal end 24 contacts the wall of the vessel 94 with the tamp 70 underneath, thereby providing tactile feedback that the outlets 25 are disposed within the puncture 90 proximal to the vessel 94 when the distal end 24 contacts the tamp 70.

A source of sealing compound 14, e.g., the dual syringe assembly 40 described above, may be prepared and connected to the side port 32 of the delivery sheath 12, e.g., via tubing 49, either before or after the delivery sheath 12 is advanced into the puncture 90. The sealing compound 99 may then be delivered through the secondary lumen 30 and the outlets 25 and into the puncture 90. The sealing compound 99 may flow radially outwardly to permeate at least partially into the tissue surrounding the puncture 90. Optionally, the delivery sheath 12 may be retracted as the sealing compound 99 is delivered, e.g., to fill the puncture 90 along its length substantially filling the tissue tract with sealing compound.

Once a desired amount of the sealing compound 99 is delivered into the puncture 90, the guidewire 60 may be maintained such that the tamp 70 continues to seal the puncture 90 from the vessel 94, e.g., for sufficient time for the sealing compound 99 to at least partially or completely cure. Thereafter (or immediately after filling the puncture 90), the delivery sheath 12 may be removed entirely from the puncture 90. Additional apparatus and methods for delivering the sealing compound 99 into the puncture 90 are disclosed in co-pending application Ser. Nos. 10/454,362 and 10/745,946, incorporated above, or in co-pending application Ser. No. 10/975,205, filed Oct. 27, 2004, the entire disclosure of which is expressly incorporated herein by reference.

Turning now to FIGS. 5A-5C, the introducer sheath 18 and sleeve 20 may then be delivered into the puncture 90 and/or through the sealing compound 99. As shown in FIG. 5A, the guidewire 60 may remain within the puncture 90 and vessel 94 after delivering the sealing compound 99. Optionally, the tamp 70 (not shown) may remain exposed and/or expanded, or the retaining sheath 50 (also not shown) may be advanced over the guidewire 60 to cover and/or collapse the tamp 70. Alternatively, the guidewire 60 may be removed from the puncture and exchanged for a separate guidewire (not shown), e.g., without a tamp, may be advanced through the puncture 90 into the vessel 94.

Turning to FIG. 5B, the introducer sheath 18, dilator 19, and sleeve 20 may then be introduced into the puncture 90, e.g., over the guidewire 60. Although not shown in FIG. 5B, the tubular member 120 (shown in FIG. 2) may be inserted along with the introducer sheath 18. For example, the guidewire 60 may be backloaded into the introducer sheath 18, e.g., by inserting the proximal end 62 of the guidewire 60 through the passage 110 (not shown, see FIG. 2) of the hub 108 into the lumen 106 (also not shown, see FIG. 2) of the sleeve 20 and directed proximally through the lumen 136 of the tubular member 120 (also not shown, see FIG. 2) (or through the lumen 136 of the dilator 19 or the lumen 86 of the introducer sheath 18, depending upon whether the tubular member 120 and/or dilator 19 are included or eliminated).

As shown in FIG. 5B, the hub 108 of the sleeve 20 may be placed against or immediately above the patient's skin 92 overlying the puncture 90. The introducer sheath 18 (and any of the dilator 19 and/or tubular member 120 carried therein) may then be advanced into the puncture 90 through the passage 110 in the hub 108. Because the sleeve 20 is disposed inside the introducer sheath 18, as the distal end 124 (not shown, see FIG. 2) of the tubular member 120 (or the distal end 134 of the dilator 19 or the distal end 84 of the introducer sheath 18) enters the passage 110, it contacts the sleeve 20 adjacent the first end 102. Further advancement of the introducer sheath 18 causes the sleeve to slide around the distal end 124 of the tubular member 120, pulling the sleeve 20 out of the lumen 106 and unfurling or everting the sleeve 20 over the distal end 124 of the tubular member 120.

As shown in FIG. 4B, as the distal end 134 of the dilator 19 and the distal end 84 of the introducer sheath 18 enter the passage 110 of the hub 108, they pass through the sleeve 20 that has everted before them. With respect to the tissue surrounding the puncture 90, the sleeve 20 unfurls or everts from the tubular member 120 as the introducer sheath 18 is advanced into the puncture 90, thereby lining the puncture 90 from the patient's skin 92 toward the vessel 94. In one embodiment, the sleeve 20 has sufficient length that the sleeve 20 substantially lines the puncture 90 through the sealing compound 99 and the second end 104 terminates adjacent the wall of the vessel 94.

Because the sleeve 20 unfurls from within the tubular member 120 as the introducer sheath 18 is advanced into the puncture 90, shear stress on the surrounding tissue, and/or on the sealing compound 99 are substantially reduced, e.g., as compared with advancing the introducer sheath 18 and/or dilator 19 through the puncture 90 without the sleeve 20. Because the introducer sheath 18 is not pushed directly along the tissue surrounding the puncture, this may substantially reduce damage to the surrounding tissue and/or to the sealing compound 99. Thus, risk of pieces of the sealing compound 99 being broken off and conveyed into the vessel 94, where they may travel downstream and cause an embolism or other damage may be substantially reduced.

Turning to FIG. 5C, the introducer sheath 18 may be advanced through the puncture 90 until the distal end 84 is disposed within the vessel 94. As the introducer sheath 18 is advanced, the second end 104 of the sleeve 20 may be completely unfurled from the tubular member 120 and exposed, e.g., within the vessel 94. In the embodiment shown, the length of the sleeve 20 is shorter than the introducer sheath 18 such that the second end 104 of the sleeve 20 is disposed proximal to the distal end 84 of the introducer sheath 18.

The dilator 19 and/or tubular member 120 may be withdrawn through the introducer sheath 18 from the puncture 90, e.g., together or successively, leaving the introducer sheath 18 and sleeve within the puncture 90. The guidewire 60 may also be removed along with, before, or after the dilator 19 and/or tubular member 120, e.g., after collapsing the tamp 70 (not shown). Alternatively, if the tamp 70 is still expanded, the guidewire 60 may be removed, causing the tamp 70 to compress to the contracted condition as it is directed into the lumen 86 of the introducer sheath 18.

Once a distal end 84 of the introducer sheath 18 is disposed within the vessel 94, one or more instruments (not shown) may be advanced through the lumen 86 into the vessel 94, e.g., to perform one or more diagnostic and/or interventional procedures within the patient's body, as is known to those skilled in the art. The sleeve 20 generally does not interfere with the introduction of such instruments, since it is located only around the introducer sheath 18. Optionally, if the sleeve 20 includes any weakened seams, the sleeve 20 may be removed from around the introducer sheath 18 to provide a conventional introducer sheath arrangement for the subsequent procedure. For example, the hub 108 may separate into two or more pieces (not shown), causing the sleeve 20 to tear or separate, e.g., along one or more predetermined seams. Thus, conventional procedures may be used without need for extra attention to the sleeve 20.

Upon completing any such procedures, the instrument(s) may be removed from the vessel 94 through the introducer sheath 18. The introducer sheath 18 and sleeve 20 (if remaining around the introducer sheath 18) may then be removed from the vessel 94 and puncture 90, e.g., simultaneously or successively. The sealing compound 99 and/or tissue may recoil sufficiently to substantially fill the puncture 90, thereby allowing and/or encouraging hemostasis to occur between the vessel 94 and puncture 90. Optionally, external pressure may be applied to the patient's skin 92 during removal of the introducer sheath 18, e.g., to further enhance sealing of the puncture 90 until hemostasis occurs.

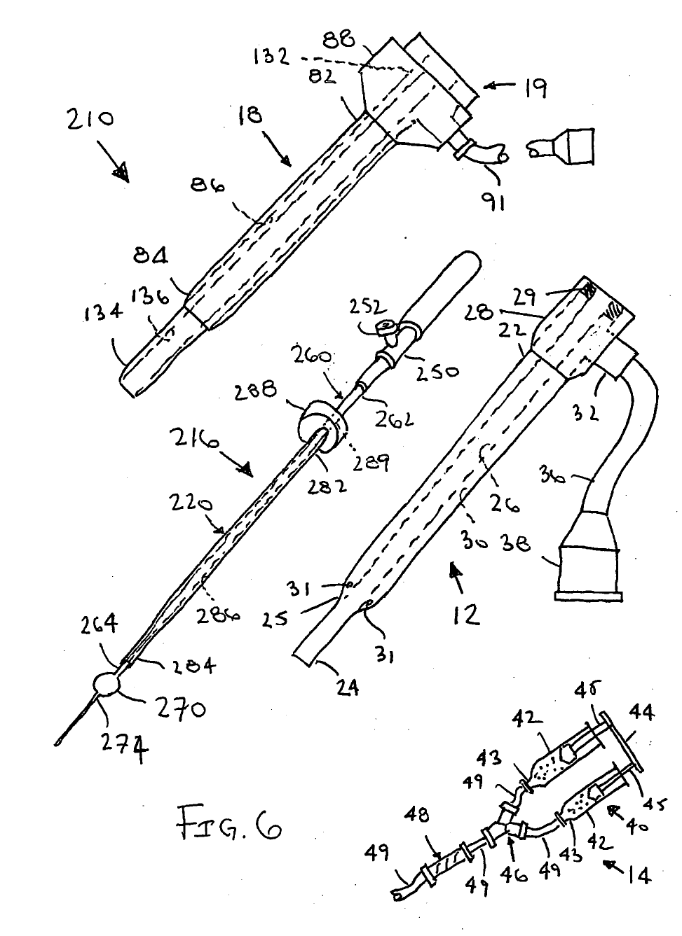

Turning to FIG. 6, another embodiment of a system 210 is shown for delivering a sleeve 220 into a puncture extending through tissue. Generally, the system 210 includes a delivery sheath 12, a source of sealing compound 14, an occlusion member 216, and an introducer or procedure sheath, such as the introducer sheath 18 described above (without the tubular member 120 and sleeve 20). Unlike the previous embodiments, the sleeve 220 is initially carried on an outer surface of the occlusion member 216, rather than within the introducer sheath 218, as described further below. Optionally, the system 210 may include other components, e.g., one or more needles, guidewires, dilators, and/or sections of tubing (not shown), similar to the previous embodiments.

Generally, the delivery sheath 12 is an elongate tubular member, similar to the previous embodiments, e.g., including a proximal end 22, a distal end 24, and a primary or guidewire lumen 26 extending between the proximal and distal ends 22, 24. The delivery sheath 12 may include one or more secondary or injection lumens 30 that extend from the proximal end 22 to one or more outlets 31 (e.g., two, as shown) in the wall of the delivery sheath 12.

The delivery sheath 12 may include a housing 28 on the proximal end 22, a side port 32 that communicates with an interior of the housing 28 and the secondary lumen 30 of the delivery sheath 12, and a section of tubing 36 extending from the side port 32. The housing 28 may include one or more seals 29 to seal the interior of the housing 28 such that sealing compound delivered into the side port 32 may be directed through the secondary lumen 30. Optionally, the housing 28 may also include one or more seals (not shown), e.g., a hemostatic seal, for sealing the primary lumen 26 while accommodating inserting one or more instruments (not shown) into the lumen 26.

The source of sealing compound 14 may include a dual syringe assembly 40 including a pair of syringe barrels 42 with outlets 43, and a plunger assembly 44 slidable into the barrels 42 to cause components therein to be delivered through the outlets 43, similar to the previous embodiments. Optionally, the delivery device 14 may include a “Y” fitting 46, a static mixer 48, and/or tubing 49, e.g., for connecting the outlets 43, the “Y” fitting 48, the mixer 48, and/or the side port 32 of the delivery sheath 12, such that the sealing components ejected out of the barrels 42 may mix before being directed into the side port 32 of the delivery sheath 12, also similar to the embodiments described above.

The occlusion member 216 is an elongate tubular member 260 including a proximal end 262, a distal end 264, and a lumen (not shown) extending between the proximal and distal ends 262, 264. The tubular member 260 may be flexible, semi-rigid, or rigid, e.g., having a uniform or variable flexibility along its length. For example, a proximal portion of the tubular member 260 may be substantially rigid, e.g., a section of hypotube (not shown), to facilitate advancing the occlusion member 216 into a puncture through tissue, while a distal portion of the tubular member 260 may be substantially flexible to facilitate insertion through a puncture into a blood vessel or other body lumen.

A balloon 270 is carried on the distal end 264 of the tubular member 260 that includes an interior communicating with the lumen of the tubular member 260. The balloon 270 is expandable from a contracted condition (not shown) to an enlarged condition, such as that shown in FIG. 6, e.g., when fluid or other inflation media is delivered through the tubular member 260 into the interior of the balloon 270. The balloon 270 may be formed from a flexible, substantially inelastic material, e.g., a nonelastomeric material, such as PET, nylon, polyethylene, polyurethane, PEBAX, and the like, that may provide a substantially noncompliant or semi-compliant balloon 270 that may expand to a predetermined size when a minimum pressure is introduced into the interior 82. Alternatively, the balloon 270 may be formed from an elastic material, such that the size of the balloon 270 in the expanded state is dependent upon the pressure or volume of fluid delivered within the interior.

In the contracted condition, the balloon 270 may conform substantially to the diameter of the tubular member 260. In one embodiment, the balloon 270 may at least partially evert in the enlarged condition, i.e., the length of the balloon 270 may be substantially smaller than the diameter. In alternative embodiments, other expandable members, e.g., a mechanically expandable or self-expanding member, such as those described above, may be provided instead of the balloon 270.

A hub 250 may be coupled to or otherwise provided on the proximal end 262 of the tubular member 260. In one embodiment, the hub 250 may be removable from the tubular member 260, e.g., using mating threads or other connectors (not shown) on the hub 250 and/or the proximal end 262 of the tubular member 260. The hub 250 may include a side port 252 that communicates with the lumen in the tubular member 260, such that a source of inflation media, e.g., a syringe containing saline (not shown), may be coupled to the side port 252 for delivering inflation media into the interior of the balloon 270.

Optionally, the occlusion member 216 may include an elongate inner member (not shown) slidable within the tubular member 260, and the hub 250 may include a piston or other mechanism (not shown) for biasing the inner member relative to the tubular member 260. For example, the inner member may be coupled to a distal end 274 of the balloon 270 and may be biased to move distally relative to the tubular member 260, e.g., to facilitate collapsing the balloon 80 when it is deflated. The piston within the hub 250 may be directed proximally when inflation media is delivered into the side port 252, thereby pulling the inner member to shorten the balloon 270 as it expands. Additional information on occlusion members that may be provided may be found in co-pending application Ser. No. 10/454,362 incorporated above.

Returning to FIG. 6, the sleeve 220 may be a relatively thin-walled substantially flexible tubular member including a first or proximal end 282, a second or distal end 284, and a lumen 286 extending therebetween, similar to the sleeves described above. In one embodiment, the sleeve 220 may be a substantially closed-walled tube that may be collapsed around the tubular member 260 of the occlusion member 216. For example, the sleeve 220 may be folded, crimped, and/or twisted about the tubular member 260 to define a collapsed state.

Optionally, the sleeve 220 may be bonded to the outer surface of the tubular member 260 and/or to itself (e.g., if folded over itself, e.g., using an adhesive that may separate when sufficient force is applied. For example, the sleeve 220 may separate from the tubular member 260 and expand towards an expanded state when an instrument, e.g., the introducer sheath 18, is advanced between the sleeve 220 and the tubular member 260, or when a fluid is injected between the sleeve 220 and the tubular member 260, as described further below.

Thus, the sleeve 220 may have a diameter or other cross-section in the expanded state that is substantially larger than the tubular member 260. In one embodiment, the sleeve 220 may be formed from a substantially inelastic material such that the sleeve 220 may assume a fixed diameter or other cross-section in the expanded state, e.g., larger than the introducer sheath 18. Alternatively, the sleeve 220 may be formed from an elastic material such that the sleeve 220 may resiliently expand to accommodate different size instruments therein.

Optionally, one or both of the inner and outer surfaces of the sleeve 220 and/or the outer surface of the tubular member 260 may include a lubricious coating. For example, if the sleeve 220 is crimped, twisted, or otherwise compressed around the tubular member 260 without being attached thereto, a lubricious coating may be provided on the inner surface of the sleeve 220 and/or on the outer surface of the tubular member 260. Such a lubricious coating may reduce friction or otherwise facilitate separation of the sleeve 220 from the tubular member 260 when the sleeve 220 is expanded, as described below.

An annular hub 288 may be attached to or otherwise provided on the proximal end 282 of the sleeve 220. The hub 288 generally includes a passage 289 therethrough that communicates with the lumen 286. The hub 288 may be expandable, e.g., such that the passage 289 may have a diameter or other cross-section larger than the outer diameter of the introducer sheath 18 when the hub 288 is expanded. The hub 288 may have an outer diameter or other cross-section that is smaller than the primary lumen 26 of the delivery sheath 12 such that the hub 288 may pass through the primary lumen 26, e.g., when the delivery sheath 12 is advanced over the occlusion member 216, as described below. Alternatively, other structures may be provided on the proximal end 282 of the sleeve 220, e.g., one or more tabs (not shown) that may lie initially against the outer surface of the occlusion member 216, instead of the hub 288. The tab(s) may be pulled transversely away from the occlusion member 216 to open and/or expand the proximal end 282 of the sleeve 220 to allow insertion of the introducer sheath 18 therein, as described further below.

Optionally, the distal end 84 of the introducer sheath 18 may be directed into the passage 289, e.g., guided by ramped surfaces or other guides (not shown) on the hub 288, and thereby into the lumen 286 of the sleeve 220, as described further below. In addition or alternatively, the hub 288 may be separable into two or more pieces (not shown) to open the proximal end 282 of the sleeve. Optionally, the sleeve 220 may include one or more seams (also not shown) such that the hub 288 and/or sleeve 220 may be separated into two or more pieces to remove the sleeve 220, similar to the embodiments described above.

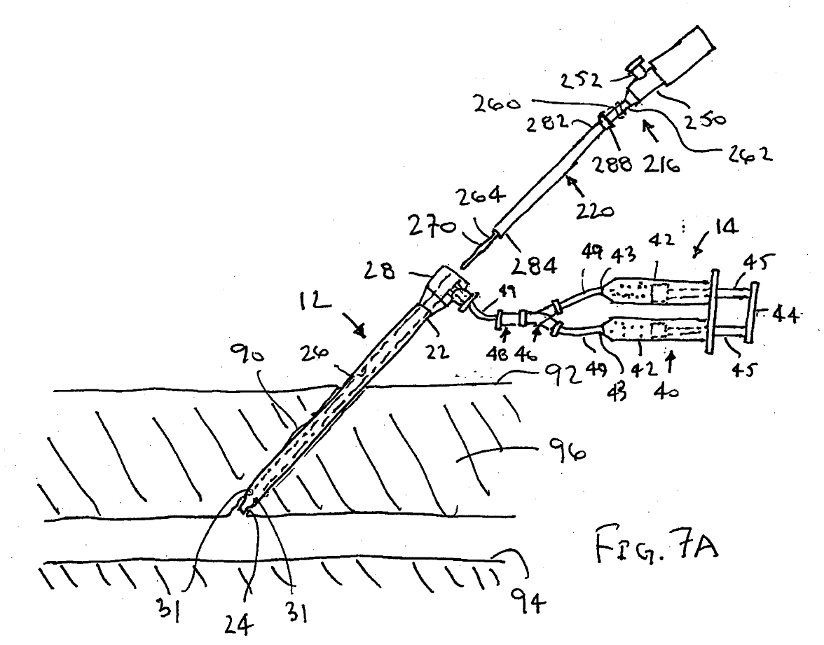

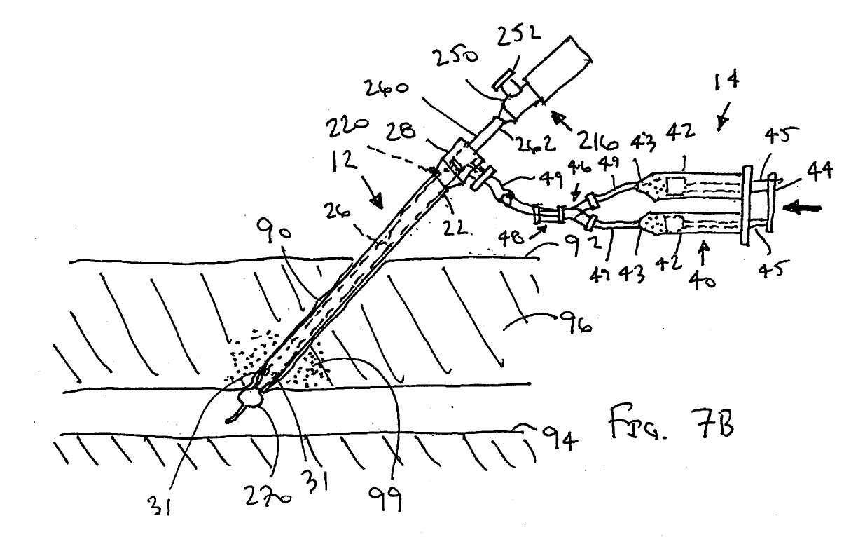

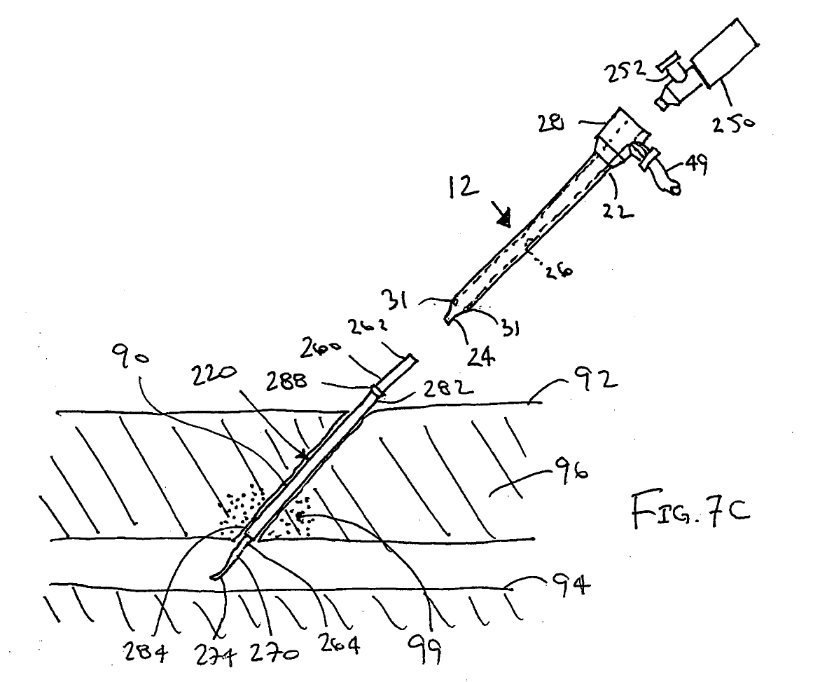

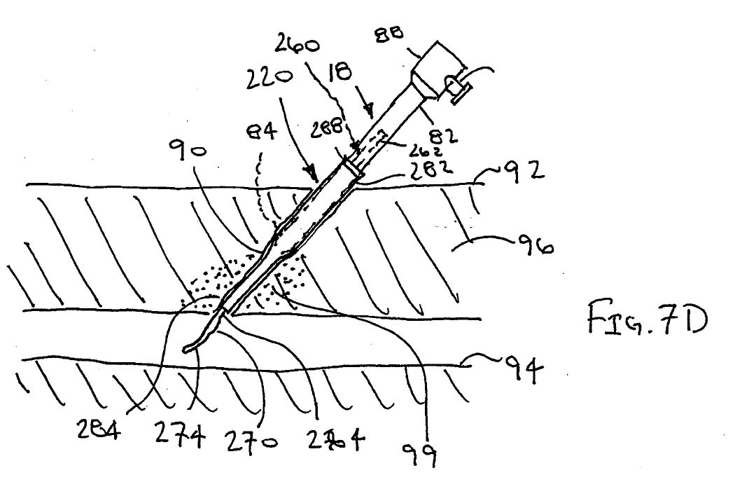

Turning to FIGS. 7A-7D, a method is shown for delivering an introducer sheath and/or sleeve, such as the introducer sheath 18 and sleeve 220 described above, into a puncture 90 extending through tissue 96, e.g., from a patient's skin 92 to a blood vessel 94. Initially, the puncture 90 may be created, e.g., using a needle (not shown), and a guidewire (also not shown) may be advanced through the needle into the vessel 94, similar to the embodiments described above. In one embodiment, the needle may be removed, and the delivery sheath 12 may be advanced over the guidewire, e.g., alone or in conjunction with one or more dilators (not shown).

As shown in FIG. 7A, once the distal end 24 of the delivery sheath 12 is disposed within the vessel 94, the guidewire and any dilators (not shown) may be removed, and the occlusion member 216 may be inserted into the puncture 90. For example, with the balloon 270 collapsed, the distal end 264 of the occlusion member 216 may be advanced through the primary lumen 26 of the delivery sheath 12 until the balloon 270 is disposed within the vessel 94. Because the sleeve 220 is collapsed around the tubular member 260, the sleeve 220 may remain unobtrusively around the tubular member 260 as the occlusion member 216 is advanced into the delivery sheath 12.

Alternatively, the occlusion member 216 may be used as the guidewire directed through the needle, and the delivery sheath 12 may be advanced over the occlusion member 216 into the puncture 90. In this alternative, the hub 250 may be separated from the tubular member 260 to allow the proximal end 262 of the tubular member 260 to be directed into the primary lumen 26 of the delivery sheath 12 before the delivery sheath 12 is advanced into the puncture 90. Optionally, the sleeve 220 may include a lubricious coating on its outer surface, e.g., to facilitate advancing the occlusion member 216 through the delivery sheath 12 and/or puncture 90 without damaging or otherwise disrupting the sleeve 220 carried on the occlusion member 216.

Turning to FIG. 7B, once the balloon 270 is within the vessel 94, the balloon 270 may be expanded and used to substantially seal the vessel 94 from the puncture 90. For example, a syringe or other source of inflation media (not shown) may be coupled to the side port 252 of the hub 250, and saline or other inflation media may be delivered through the tubular member 260 into the interior of the balloon 270, causing the balloon 270 to expand to the expanded state. If the hub 250 is previously separated from the tubular member 260, the hub 250 may be attached to the proximal end 262 of the tubular member 260 to allow the inflation media to be delivered via the side port 252 into the tubular member 260 and the interior of the balloon 270. Alternatively, other selectively expandable members may be provided on the distal end 264 of the tubular member 260 instead of the balloon 270, such as the expanding mesh or expandable frame (not shown) described above, and the expandable member may be expanded within the vessel 94.

With the balloon 270 (or other expandable member) expanded, the occlusion member 216 may be partially retracted from the vessel 94, e.g., by pulling the hub 250, until the balloon 270 contacts the distal end 24 of the delivery sheath 12 (providing a first tactile feedback). The occlusion member 216 may then be pulled further until the balloon 270 contacts the wall of the vessel 94 (providing a second tactile feedback), thereby partially retracting the delivery sheath 12 back into or above the puncture 90. Alternatively, the balloon 270 may be directed against the wall of the vessel 94 before the delivery sheath 12 is advanced fully into the puncture 90, e.g., until the distal end 24 of the delivery sheath 12 contacts the vessel 94 with balloon 270 underneath.

A source of sealing compound 14, e.g., the dual syringe assembly 40 described above, may be connected to the side port 32 of the delivery sheath 12, and sealing compound 99 may be delivered through the delivery sheath 12 into the puncture 90. Optionally, the delivery sheath 12 may be retracted as the sealing compound 99 is delivered, e.g., similar to the embodiments described above. The occlusion member 216 may be maintained such that the balloon 270 continues to seal the puncture 90 from the vessel 94, e.g., for sufficient time for the sealing compound 99 to at least partially or completely cure.

Thereafter, as shown in FIG. 7C, the delivery sheath 12 may be removed entirely from the puncture 90, leaving the tubular member 260 and sleeve 220 within the puncture 90. For example, the balloon 270 may be collapsed, e.g., by evacuating the inflation media from the balloon 270 and/or by removing the hub 250 from the proximal end 262 of the tubular member 260. The hub 250 may be removed from the tubular member 260 (if not already), and the delivery sheath 12 may then be withdrawn from the puncture 90 around the tubular member 260 and sleeve 220.

Turning to FIG. 7D, the introducer sheath 18 may then be delivered into the puncture 90 and/or through the sealing compound 99 via the sleeve 220. As shown, the distal end 84 of the introducer sheath 18 may then be advanced over the proximal end 262 of the tubular member 260, and inserted into the proximal end 282 of the sleeve 220, i.e., such that the introducer sheath 18 is disposed between the sleeve 220 and the tubular member 260. Optionally, the hub 288 may be expanded or tabs or others structures (not shown) on the proximal end 282 of the sleeve 220 may be manipulated to separate and/or expand the proximal end 282 of the sleeve 220 to accommodate the introducer sheath 18.

As the introducer sheath 18 is advanced distally, the sleeve 220 may separate and expand away from the tubular member 260, thereby pushing the surrounding tissue and/or sealing compound radially away from the introducer sheath 18. The distal end 84 of the introducer sheath 18 may exit the distal end 284 of the sleeve 220 and enter the vessel 94, thereby providing an arrangement similar to that shown in FIG. 5C.

Alternatively, the occlusion member 216 may be removed from the puncture 90, leaving the sleeve 220 behind before the introducer sheath 18 is advanced into the puncture 90. For example, the occlusion member 216 may be twisted about its longitudinal axis to cause the sleeve 220 to separate from the tubular member 260. Alternatively, fluid may be delivered into the lumen 286 of the sleeve 220 to cause the sleeve 220 to expand, and thereby separate from the tubular member 260. For example, the hub 288 may include a side port (not shown) that may be coupled to a syringe or other source of fluid (not shown) such that fluid from the syringe may be directed between the sleeve 220 and the tubular member 260. The introducer sheath 18 (optionally with one or more dilators 19) may then be advanced through the sleeve 220 and into the puncture 90 until the distal end 84 enters the vessel 94.

Because the introducer sheath 18 slides along the inner surface of the sleeve 220, the surrounding tissue and/or sealing compound may be substantially protected from shear stresses that may otherwise damage the tissue or break off pieces of the sealing compound. In the embodiment shown, the length of the sleeve 220 is shorter than the introducer sheath 18 such that the second end 284 of the sleeve 220 is disposed proximal to the distal end 84 of the introducer sheath 18.

The dilator (if provided) may be withdrawn through the introducer sheath 18 from the puncture 90, leaving the introducer sheath 18 and sleeve within the puncture 90. The occlusion member 260 may also be removed if not already removed from the introducer sheath 18. One or more instruments (not shown) may be advanced through the lumen 86 into the vessel 94, e.g., to perform one or more diagnostic and/or interventional procedures within the patient's body, as is known to those skilled in the art. The sleeve 220 generally does not interfere with the introduction of such instruments, since it is located only around the introducer sheath 18.

Optionally, if the sleeve 220 includes any weakened seams, the sleeve 220 may be removed from around the introducer sheath 18 to provide a conventional introducer sheath arrangement for the subsequent procedure. For example, the hub 288 may separate into two or more pieces (not shown), causing the sleeve 220 to tear or separate, e.g., along one or more predetermined seams. Thus, conventional procedures may be used without need for extra attention to the sleeve 20.

Upon completing any such procedures, the instrument(s) may be removed from the vessel 94 through the introducer sheath 18. The introducer sheath 18 and sleeve 220 (if remaining around the introducer sheath 18) may then be removed from the vessel 94 and puncture 90, e.g., simultaneously or successively. As described above, the sleeve 220 may include a lubricious coating on its outer surface, e.g., to minimize the risk of the sealing compound adhering to the sleeve 220 and/or to facilitate removing the sleeve 220 from the puncture 90.

The sealing compound 99 and/or tissue may recoil sufficiently to substantially fill the puncture 90, thereby allowing and/or encouraging hemostasis to occur between the vessel 94 and puncture 90. Optionally, external pressure may be applied to the patient's skin 92 during removal of the introducer sheath 18, e.g., to further enhance sealing of the puncture 90 until hemostasis occurs.

While the invention is susceptible to various modifications, and alternative forms, specific examples thereof have been shown in the drawings and are herein described in detail. It should be understood, however, that the invention is not to be limited to the particular embodiments or methods disclosed, but to the contrary, the invention is to cover all modifications, equivalents and alternatives falling within the scope of the appended claims.

1. Apparatus for introducing an instrument into a body lumen of a patient through a puncture extending from the patient's skin to the body lumen, comprising:
a guidewire;
a sealing compound in a form that can be delivered into the puncture around the guidewire; and
an instrument introducer assembly comprising
a tubular sheath having a proximal end, a distal end shaped for insertion into the puncture, and a lumen extending from the proximal end to an opening in the distal end, and
a flexible, thin-walled sleeve comprising first and second ends, and a hub on the first end disposed adjacent the distal end of the sheath, the second end extending into the distal opening and lumen of the sheath, the hub being slidable along an exterior of the sheath for drawing the sleeve out of the distal sheath opening and along the sheath exterior.

2. The apparatus of claim 1, wherein the sleeve is expandable from a contracted condition when disposed within the lumen of the sheath to an expanded condition when removed from the sheath lumen.

3. The apparatus of claim 2, wherein the sleeve is collapsed into the contracted condition.

4. The apparatus of claim 1, wherein the sleeve is substantially nonporous.

5. The apparatus of claim 2, wherein the sleeve comprises substantially inelastic material, such that the sleeve defines a predetermined diameter in the expanded condition.

6. The apparatus of claim 1, wherein the sleeve comprises a lubricious coating on at least one of an inner surface and an outer surface.

7. The apparatus of claim 1, wherein the sleeve is separable along one or more seams extending between the first and second ends.

8. The apparatus of claim 1, the instrument introducer assembly further comprising a tubular dilator slidably disposed within the lumen of the sheath, the second end of the sleeve being disposed within a lumen of the dilator.

9. The apparatus of claim 1, further comprising means for delivering the sealing compound into the puncture around the guidewire.

10. The apparatus of claim 1, further comprising a delivery assembly for delivering the sealing compound into the puncture around the guidewire, the delivery assembly including a tubular sheath having a first lumen sized for allowing passage there through of the guidewire, and a second lumen at least partially surrounding and isolated from the first lumen, the second lumen in communication with an inlet port for receiving the sealing compound and one or more outlet ports arranged to dispense the sealing compound within the puncture around the guidewire.

11. A method for lining a puncture extending from a patient's skin to a body lumen using a tubular sheath and a flexible, thin-walled sleeve disposed within a lumen of the tubular sheath, the sleeve comprising a hub on a first end thereof adjacent a distal end of the sheath and a second end disposed within the lumen of the sheath, the method comprising:
placing a guide wire through the puncture from the patient's skin into the body lumen;
introducing a sealing compound into the puncture around at least a portion of the guide wire;
placing the hub of the sleeve adjacent the patient's skin; and
advancing the sheath into the puncture over the guidewire, while maintaining the hub adjacent the patient's skin, causing the sleeve to be deployed from the lumen of the tubular sheath and cover an exterior of the sheath to thereby line the puncture as the sheath is advanced into the puncture.

12. The method of claim 11, wherein the sheath is advanced into the puncture until the distal end of the sheath is disposed within the body lumen, the sleeve having sufficient length to extend from the hub adjacent the patient's skin to the body lumen.

13. The method of claim 12, further comprising introducing an instrument through the sheath into the body lumen to perform a procedure within the patient's body.

14. The method of claim 13, further comprising removing the sheath from the puncture after completing the procedure.

15. The method of claim 14, wherein the sleeve is removed from the puncture substantially simultaneously with removing the sheath from the puncture.

16. The method of claim 14, wherein the sleeve is removed from the puncture prior to the sheath.

17. The method of claim 16, wherein the sleeve is separated along one or more seams as it is removed from the puncture to allow the sleeve to be removed from around the sheath.

18. The method of claim 11, further comprising loading the guidewire through the lumen of the sheath, the sheath being advanced over the guidewire when the sheath is introduced into the puncture.

19. The method of claim 11, wherein the guidewire extends through the sleeve when the guidewire is loaded through the lumen of the sheath.

20. A system for introducing an instrument into a body lumen of a patient through a puncture extending from the patient's skin to the body lumen, comprising:
a sealing compound delivery assembly including
a sealing compound, and
a tubular sheath having a first lumen sized for allowing passage there through of a guidewire, and a second lumen at least partially surrounding and isolated from the first lumen, the second lumen in communication with an inlet port for receiving the sealing compound and one or more outlet ports for dispensing the sealing compound within the puncture while a guidewire is positioned in the first lumen; and
an instrument introducer assembly including
a tubular sheath having a proximal end, a distal end shaped for insertion into the puncture, and a lumen extending from the proximal end to an opening in the distal end, and
a flexible, thin-walled sleeve comprising first and second ends, and a hub on the first end disposed adjacent the distal end of the sheath, the second end extending into the distal opening and lumen of the sheath, the hub being slidable along an exterior of the sheath for drawing the sleeve out of the distal sheath opening and along the sheath exterior.
